(19) United States
(12) Patent Application Publication
(10) Pub. No.: US 2025/0213861 A1
(43) Pub. Date: Jul. 3, 2025

(54) SKIN TREATMENT DEVICE, METHOD FOR SKIN TREATMENT, OUTPUT CONTROL METHOD, AND READABLE STORAGE MEDIUM

(71) Applicant: YA-MAN LTD., Tokyo (JP)

(72) Inventors: Masatake KISHI, Tokyo (JP); Asako TANAKA, Tokyo (JP)

(21) Appl. No.: 18/795,281

(22) Filed: Aug. 6, 2024

(30) Foreign Application Priority Data
Dec. 27, 2023 (JP) 2023-220654
Apr. 26, 2024 (JP) 2024-072413
May 14, 2024 (JP) 2024-078505
Jun. 28, 2024 (JP) 2024-104673

(51) Int. Cl. A61N 1/36

(52) U.S. Cl. CPC A61N 1/36014

(57) ABSTRACT

Disclosed is a skin processing device, a method for skin treatment, an output control method, and a storage medium, simultaneously applying an electric stimulation and a warming action to a skin surface by combining an AC stimulation of 40 kHz˜250 kHz with a distance between electrodes of 0.5 mm˜60 mm related to a pair of the electrodes that apply the AC stimulation to skin.

### CROSS REFERENCE TO RELATED APPLICATIONS

This application claims the benefit of Japanese application No. 2023-220654 filed on Dec. 27, 2023, Japanese application No. 2024-72413 filed on Apr. 26, 2024, Japanese application No. 2024-78505 filed on May 14, 2024, Japanese application No. 2024-104673 filed on Jun. 28, 2024, and Japanese Patent Application No. 2024-104673 filed on Jun. 28, 2024, the entire contents of which are incorporated by reference herein.

### TECHNICAL FIELD

The present disclosure relates to a skin treatment device, a method for skin treatment, an output control method, and a readable storage medium.

### BACKGROUND ART

There has been known a technology that activates fibroblasts existing in the skin by stimulating them with a physical stimulation generating circuit (e.g. ultrasonic oscillation circuit, low frequency generating circuit, heat generating circuit, light wavelength oscillation circuit) to promote the production of collagen and estrane.

### PRIOR ART DOCUMENT

### Patent Document

Patent Document 1: JP 2005-334517A

### SUMMARY OF THE INVENTION

### Problems to be Solved by the Invention

However, the above-mentioned conventional technology targets a variety of physical stimulation, and it is difficult to improve the penetration effect of active ingredients to the skin and the effect related to skin lifting.

The present disclosure therefore aims to improve the penetration effect of active ingredients to the skin and the effect related to skin lifting.

### Means for Solving the Problems

Provided according to one aspect of the present disclosure is a method for skin treatment, which is simultaneously applying an electrical stimulation and a warming action to the skin surface by combining an AC (alternating current; the same below) stimulation of 40 kHz to 250 kHz with a distance between electrodes of 0.5 mm to 60 mm related to a pair of electrodes that apply the AC stimulation to the skin, and the like.

Provided according to another aspect of the present disclosure is a skin treatment device, which is simultaneously applying an electrical stimulation and a warming action to the skin surface by combining an AC stimulation of 40 kHz to 250 kHz with a distance between electrodes of 0.5 mm to 60 mm according to a pair of electrodes that apply the AC stimulation to the skin.

Provided according to another aspect of the present disclosure is an output control method, which is controlling so as to simultaneously apply an electrical stimulation and a warming action to the skin surface by combining an AC stimulation of 40 kHz to 250 kHz with a distance between electrodes of 0.5 mm to 60 mm related to a pair of electrodes that apply the AC stimulation to the skin.

Provided according to another aspect of the present disclosure is a readable storage medium that stores a program for implementing an output control method of the skin treatment device, which controls so as to simultaneously apply an electrical stimulation and a warming action to the skin by combining an AC stimulation of 40 kHz to 250 kHz with an distance between electrodes of 0.5 mm to 60 mm related to a pair of electrodes that apply the AC stimulation to the skin.

### Effects of the Invention

According to the present disclosure, improvement of the penetration effect of active ingredients to the skin and the effect related to skin lifting can be performed.

### BRIEF DESCRIPTION OF THE DRAWINGS

FIG. 1 is a perspective view showing the appearance of a skin treatment device according to this embodiment.

FIG. 2 is a diagram illustrating the head part of the skin treatment device in FIG. 1, FIG. 2A is a front view showing the arrangement of a plurality of electrodes and a plurality of outer edge electrodes, and FIG. 2B is a front view of the electrode.

FIG. 3 is a front view illustrating an example of the arrangement of a plurality of electrodes, FIG. 3A is showing the arrangement of the electrodes, that are adjacent to one another, FIG. 3B is showing the arrangement of the electrodes, that are contact with one another, and FIG. 3C is showing the arrangement of the electrodes, that are integrated with one another.

FIG. 4 is a front view illustrating the linear parallel output area and the equally spaced output area of the electrode in FIG. 2B.

FIG. 5 is a perspective view showing the exterior of a skin treatment device according to another embodiment.

FIG. 6 is an explanatory diagram of a control device built in the skin treatment device according to this embodiment.

FIG. 7 is a diagram showing an example of the hardware configuration of the control device.

FIG. 7A is a flowchart showing an example of the operation of the control device.

FIG. 8 is an explanatory diagram of two examples of AC waveforms.

FIG. 9 is an explanatory diagram visually showing the preferred values of each parameter.

FIG. 10 is a table showing the results of sensory rating.

FIG. 11 is a table showing the results of thermography measurement.

FIG. 12A is an explanatory diagram visually showing the preferred values of each parameter.

FIG. 12B is an explanatory diagram visually showing the preferred values of each parameter.

FIG. 12C is an explanatory diagram visually showing the preferred values of each parameter.

FIG. 12D is an explanatory diagram visually showing the preferred values of each parameter.

FIG. 12E is an explanatory diagram visually showing the preferred values of each parameter.

FIG. 12F is an explanatory diagram visually showing the preferred values of each parameter.

FIG. 12G is an explanatory diagram visually showing the preferred values of each parameter.

FIG. 12H is an explanatory diagram visually showing the preferred values of each parameter.

FIG. 12I is an explanatory diagram visually showing the preferred values of each parameter.

FIG. 13 is an explanatory diagram of the voltage in the test conditions.

FIG. 13A is an explanatory diagram of the voltage in the test conditions for the distance between electrodes.

FIG. 13AA indicates a voltage lower limit for balancing a warmth feeling and the muscle stimulation when the distance of electrodes is 0.5 mm.

FIG. 13AB indicates a voltage lower limit for balancing a warmth feeling and the muscle stimulation when the distance of electrodes is 5 mm.

FIG. 13AC indicates a voltage lower limit for balancing a warmth feeling and the muscle stimulation when the distance of electrodes is 10 mm.

FIG. 14 is an explanatory diagram of the electrode configuration of another embodiment.

FIG. 15 An explanatory diagram of the head part configuration of another embodiment.

FIG. 16 An explanatory diagram of the variation of the distance between electrodes in another embodiment.

FIG. 17 An explanatory diagram of the variation of the distance between electrodes in another embodiment.

FIG. 18 An explanatory diagram of the variation of the distance between electrodes in another embodiment.

### MODES FOR CARRYING OUT THE INVENTION

Exemplary embodiments will now be described in detail with reference to the accompanying drawings.

### Embodiment 1

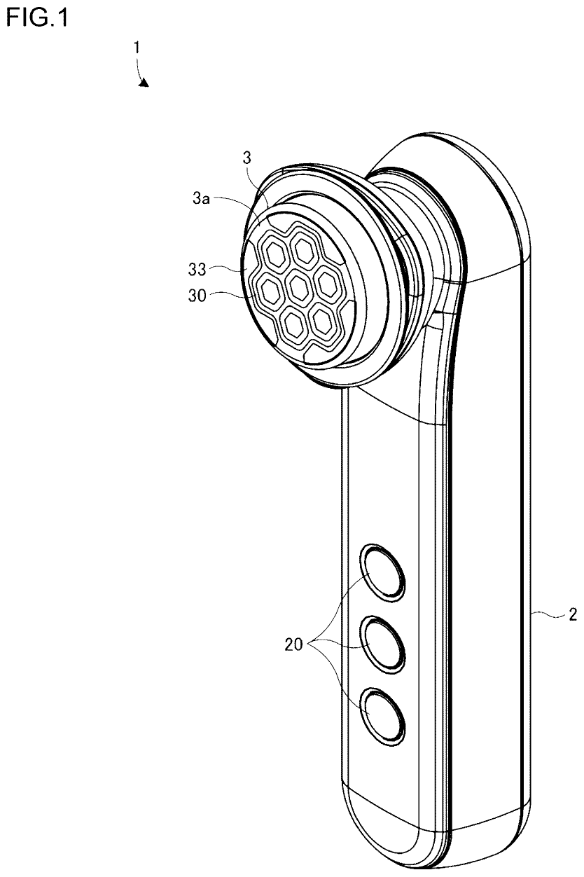

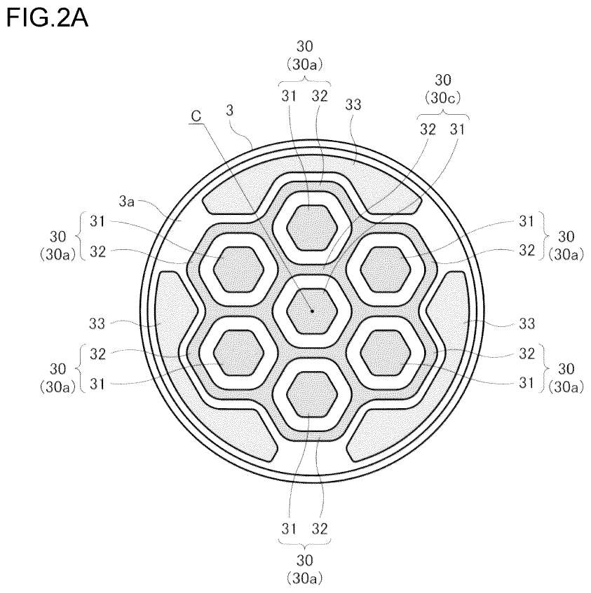

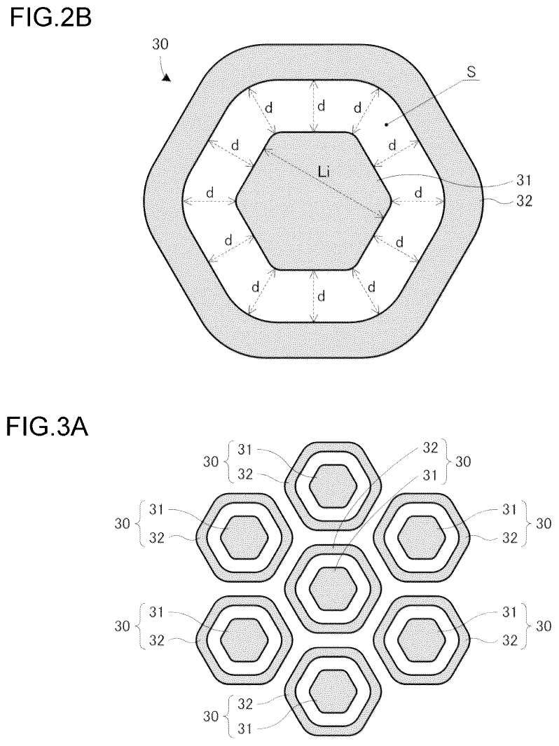

FIG. 1 is a perspective view showing the appearance of a skin treatment device 1 according to this embodiment as an example of a specific configuration of a skin treatment device. FIG. 2 is an explanatory diagram illustrating a head part 3 of the skin treatment device 1 according to this embodiment.

The skin treatment device 1 according to this embodiment is in the form of a facial beautifier and is configured to apply an action related to beauty to the skin of user's face part. However, in a modified example, in addition to or instead of the user's face part, it may be configured to apply a same effect to a part other than the user's face part. Furthermore, the skin treatment device 1 may be used to apply a different effect (e.g. an effect of promoting transdermal absorption of pharmaceuticals) from the beauty-related effect.

The beauty-related effect is optional, and it may include the elimination of sagging, tightening, fat burning, lifting, making the face smaller, improving the firmness, luster, and moisture of the skin, or any combination of two or more of the above. Furthermore, the beauty-related effect may be a quantifiable effect or an unquantifiable effect.

The skin treatment device 1 is configured to apply the beauty-related effect to the user's skin by applying various outputs through a plurality of electrodes that contact with the user's skin.

The skin treatment device 1 according to this embodiment is portable type that can be held by user's hand, but it may be applied to a movable type that is movably supported on a fixed device via an arm and the like.

The skin treatment device 1 according to this embodiment includes a holding part 2 and a head part 3. In this case, the user can apply various outputs from the skin treatment device 1 to a desired region by holding the holding part 2 and contacting the head part 3 to the desired region of the user's face or the face of another person (e.g., a patient).

The holding part 2 has a shape that is easily held by the user's hand. The holding part 2 may include a user interface 20 that includes various buttons such as a power on/off button, a mode switching button, and an intensity adjustment button. The various buttons may be mechanical buttons or touch switches. The holding part 2 may also be provided with a display part (not illustrated) that displays the state of the skin treatment device 1 and the like. The holding part 2 may also be provided with an electrode (not illustrated) that touches the user's hand.

The head part 3 is provided at the end of the holding part 2. The head part 3 may be fixed to the holding part 2, may be detachable, or may be movable to the holding part 2.

The head part 3 can be contacted with the user's skin and has a shape suitable for be contacted with the user's skin. The head part 3 may have, for example, a substantially planar contact surface 3a (including a curved surface with a relatively large radius of curvature). The contact surface 3a is a plane whose extension direction (basic surface) can be approximated to a substantially straight line in a side view. The shape of the contact surface 3a in a front view (i.e., the shape when viewed in a direction perpendicular to the contact surface 3a) may be in optional shape, such as a rectangle, a circle, an oval, or a polygon. In this embodiment, the shape of the contact surface 3a when viewed from the front is, for example, circular, as shown in FIG. 2A. The center of the contact surface 3a of the head part 3 when viewed from the front (i.e., the center of gravity when viewed in a direction perpendicular to the contact surface 3a) is referred to as the “center C of the contact surface 3a”

A plurality of electrodes groups are arranged for each attribute, specifically, a first electrode group and a second electrode group are arranged.

The first electrode group includes a plurality of electrodes 30 arranged in an array on the contact surface 3a. The second electrode group includes a plurality of outer edge electrodes 33 arranged on the contact surface 3a in a mutually rotationally symmetrical form around the center C of the contact surface 3a (which is also the center of the first electrode group) in a manner that surround the plurality of electrodes 30. These electrodes 30 and the outer edge electrodes 33 are formed so as to be easily in contact with the user's skin, and may be flush with the basic surface of the contact surface 3a of the head part 3, or may be slightly projecting from the basic surface of the contact surface 3a of the head part 3.

In this embodiment, the head part 3 has seven electrodes 30 as the first electrode group, but the number of electrodes 30 in the first electrode group is not limited to seven, may be any number greater than or equal to one. In this embodiment, the head part 3 has three outer edge electrode 33, but the number of the outer electrodes 33 as the second electrodes group is not limited to 3, may be any number greater than or equal to 2.

Each of the plurality of electrodes 30 has an inner electrode 31 and an outer electrode 32 that is spaced apart from the inner electrode 31 and surrounds the inner electrode 31. The inner electrode 31 and the outer electrode 32 of each of a plurality of electrodes 30 form a pair of electrodes for applying an output waveform of a predetermined frequency having, for example, an action related to beauty (specifically, effects related to sagging, wrinkles, and lifting, which will be described later) to the user's skin.

That is, in the first electrode group, the inner electrode 31 and the outer electrode 32 of each of the plurality of electrodes 30 form a pair to generate a desired output waveform. In this case, the output waveform is arbitrary and may be, for example, an AC waveform or a pulse-like DC (direct current; the same below) waveform. Preferred examples of the frequency band of the AC output waveform will be described later. Also, some examples of output waveforms realized by pairing the inner electrode 31 and the outer electrode 32 will be described later. In addition, in the description of the present invention, unless otherwise specified, high frequency refers to a frequency band greater than 10 kHz, and low frequency refers to a frequency band of 10 kHz or less.

By arranging the plurality of outer edge electrodes 33 constituting the second electrode group in a manner that surround at least a part of the plurality of electrodes 30 constituting the first electrode group, it is possible to configure it so that a synergistic effect is achieved between the action provided by the first electrode group and the action provided by the second electrode group. In addition, by making the shape and arrangement of the a plurality of outer electrodes 33 constituting the second electrode group so as to along to the overall shape of the assembly of the a plurality of electrodes 30 constituting the first electrode group, it is possible to use the contact surface 3a of the head part 3 without waste and secure an appropriate (in other words, sufficient) space for the electrodes constituting the second electrode group. This makes it possible to prevent discomfort caused by a strong stimulation felt when a low-frequency current is passed through an electrode with a small area.

The plurality of outer electrodes 33 form a pair of electrodes for applying an output waveform of a predetermined frequency having, for example, an action related to beauty (specifically, an electrical muscle stimulation action) to the user's skin.

That is, in the second electrode group, the outer electrodes 33 are paired together to generate a desired output waveform. In this case, the output waveform may be optional, and may be, for example, an AC waveform or a pulse-like DC waveform. In this case, the frequency band of the output waveform may be optional, for example, a high frequency or low frequency that has an electrical muscular stimulation action. Some examples of output waveforms that are realized by pairing the outer electrodes 33 together will be described later.

In this embodiment, each of the plurality of electrodes 30 has an inner electrode 31 with an outer periphery formed in a regular hexagon, and the inner and outer peripheries of the outer electrode 32 are also formed in a regular hexagon, and the outer electrode 32 is formed in a regular hexagonal band ring shape in a manner that surround the inner electrodes 31 at a distance from the inner electrodes 31. That is, the outer electrode 32 are arranged outside of the outer circumference of the inner electrodes 31 (in other words, radially outward) so that the center of the inner electrodes 31 (center of gravity position in front view; same below) and the center of the outer electrode 32 (center of gravity position in front view; same below) coincide with each other.

In this embodiment, each of the a plurality of electrodes 30 is formed so that the shape of the outer periphery of the inner electrode 31 and the shapes of the inner and outer peripheries of the outer electrode 32 have a regular hexagon with rounded corners, so that the dimension d between the outer periphery of the inner electrode 31 and the inner periphery of the outer electrode 32 is constant throughout the entire inter-electrode area S between the inner electrode 31 and the outer electrode 32 (see FIG. 2B). In this case, the symmetry and uniformity of the distance from the inner electrode 31 to the outer electrode 32 realizes uniform electrical application with suppressed electrical bias between the inner electrode 31 and the outer electrode 32. However, the shape of the outer peripheral edge of the inner electrode 31 and the shapes of the inner peripheral edge and outer peripheral edge of the outer electrode 32 may be formed to have shapes that are not rounded.

In this embodiment, all the plurality of electrodes 30 have the same shape. However, some of the plurality of electrodes 30 may have different shapes (in other words, some of the electrodes may have the same shape), or all the plurality of electrodes 30 may have shapes different from one another. In other words, the plurality of electrodes 30 may all be arranged to have the same shape, or the electrodes with two or more different shapes that are different from each other.

One electrode 30 (reference number 30c in FIG. 2A) is arranged in a manner that the center of the inner electrode 31 coincides with the center C of the contact surface 3a. Furthermore, six electrodes 30 (reference number 30a in FIG. 2A) are arranged around the electrode 30 (reference number 30c in FIG. 2A) arranged at the center C of the contact surface 3a, on a circumference centered at the center C of the contact surface 3a, in a manner that is equally spaced from one another.

The dimension Li between facing edges of the outer periphery of the inner electrode 31 is not limited to a specific value, just as an example, it may be set to any value within the range of about 2 to 5 mm.

The dimension between the centers of the inner electrodes 31 of adjacent electrodes 30 is not limited to a specific value, just as an example, it may be set to any value within the range of about 4 to 12 mm.

In this embodiment, as described above, each of the a plurality of electrodes 30 is configured so that the shape of the outer peripheral edge of the inner electrode 31 is formed in a regular hexagon (more specifically, a regular hexagon with rounded corners; same below), and the shapes of the inner and outer peripheral edges of the outer electrode 32 are regular hexagons, and the inner electrode 31 and the outer electrode 32 are combined so that the center of the inner electrode 31 and the center of the outer electrode 32 coincide with each other.

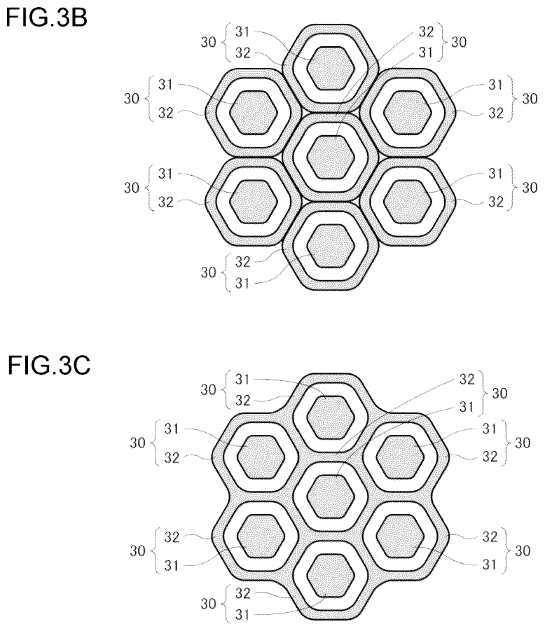

Moreover, the plurality of electrodes 30 are arranged in an array such that the plurality of outer electrodes 32 are adjacent to one another (see FIG. 3A), in contact with one another (see FIG. 3B), or integrated with one another (see FIG. 3C; this embodiment). In adjacent electrodes 30, the outer electrodes 32 may be integrated with one another (in other words, they may overlap or be common), but are arranged so as not to intersect with each other.

In this embodiment, the plurality of electrodes 30 are arranged in a manner that at least a part of the outer electrodes 32 of adjacent electrodes 30 are common, that is, in the manner shown in FIG. 3C. In this case, the outer electrodes 32 are formed in a mesh shape when viewed from the front, specifically in a honeycomb shape. Furthermore, the outer edge of the outer electrodes 32 is formed in a regular hexagon, and the plurality of electrodes 30 are arranged so that at least a part of the outer electrodes 32 of adjacent electrodes 30 are integrated (in other words, overlapping or common), so that there are no gaps between the electrodes 30 (in other words, no wasted space), and the number and arrangement of the electrodes 30 are adjusted according to the size and shape of the contact surface 3a, and the electrodes 30 can be arranged so as to fill the entire surface of the contact surface 3a.

Furthermore, by assembling a plurality of electrodes 30, each of which is made up of an inner electrode 31 and an outer electrode 32 surrounding it, to form an electrode assembly, it is possible to increase the expandability and freedom of arrangement of the electrodes, and it becomes possible to freely adjust the shape of the entire electrode assembly according to the region to which the beauty-related effect is to be applied. Specifically, for example, the shape of the entire electrode assembly may be a shape that fills an approximately circular area as in this embodiment, a shape that fills an approximately oval area, a shape that fills an approximately rectangular area, or even a shape that fills an approximately gourd-shaped area.

By configuring a pair of electrodes with the inner electrode 31 and an outer electrode 32 that is separated from the inner electrode 31 and surrounding the inner electrode 31, and changing the size of the inner electrode 31 and the outer electrode 32 or the width of the outer electrode 32, the distance between a pair of electrodes (i.e. dimension d between outer peripheral edges of the inner electrode 31 and inner peripheral edge of the outer electrode 32) is adjusted to the arbitrary value. The preferred range of dimension d (also referred to as “distance between electrodes”) between the outer edge of the inner electrode 31 and the inner peripheral edge of the outer electrode 32 will be described later.

In the electrode 30 both the shape of outer peripheral edge of the inner electrode 31 and inner peripheral edge of the outer electrode 32 have linear and mutually parallel part SP. In this way, uniform electrical application that electrical bias is well suppressed is realized.

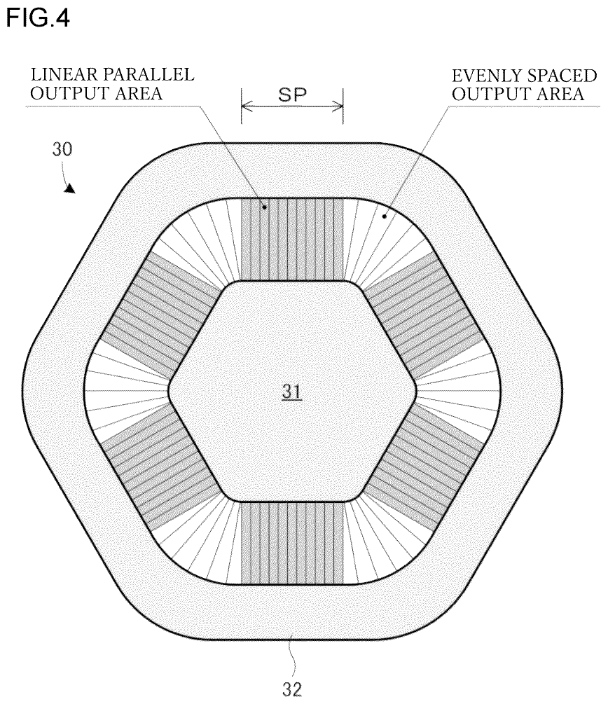

In this embodiment, as shown in FIG. 4, both the shape of the outer peripheral edge of the inner electrode 31 and the inner peripheral edge of the outer electrode 32 of the electrode 30 have linear and mutually parallel part SP. And in the area between the inner electrode 31 and the outer electrode 32 in this parallel part SP (the “linear parallel output area” that is the dark gray shaded area in FIG. 4), uniform electrical application in which electrical bias is suppressed more well is realized. Moreover, the shape of the outer peripheral edge of the inner electrode 31 and the shape of the inner peripheral edge of the outer electrode 32 are formed in a regular hexagon with rounded corners, so that the dimension d between the outer edge of the inner electrode 31 and the inner peripheral edge of the outer electrode 32 is constant throughout the electrode area S between the inner electrode 31 and the outer electrode 32, and in the area between the inner electrode 31 and the outer electrode 32 in the rounded corner portion (the “evenly spaced output area” that is the portion between the straight parallel output regions in FIG. 4), uniform electrical application in which electrical bias is suppressed is realized.

The ratio of the area of the outer electrode 32 to the area of the inner electrode 31 of each electrode 30 (referred to as “ratio of the inner and outer electrode areas”) is preferably within a predetermined range. The ratio of the inner and outer electrode areas is preferably 0.8 or more and 1.2 or less, more preferably 0.9 or more and 1.1 or less, more preferably 0.95 or more and 1.05 or less, and most preferably 1.0. By setting the ratio of the inner and outer electrode areas to an appropriate range, good electrical application between the inner electrode 31 and the outer electrode 32 is realized.

The ratio of the area of the inter-electrode area S between the inner electrode 31 and the outer electrode 32 to the sum of the area of the inner electrode 31 and the area of the outer electrode 32 of the plurality of electrodes 30 (referred to as “ratio of the inter-electrode area to the electrode area”) is preferably within a predetermined range. The ratio of the inter-electrode area to the electrode area is preferably 0.6 or more and 1.6 or less, more preferably 0.6 or more and 1.2 or less, more preferably 0.7 or more and 1.1 or less, and most preferably 0.9 or more and 1.0 or less. By setting ratio of the inter-electrode area to the electrode area to an appropriate range, good electrical application between the inner electrode 31 and the outer electrode 32 is realized.

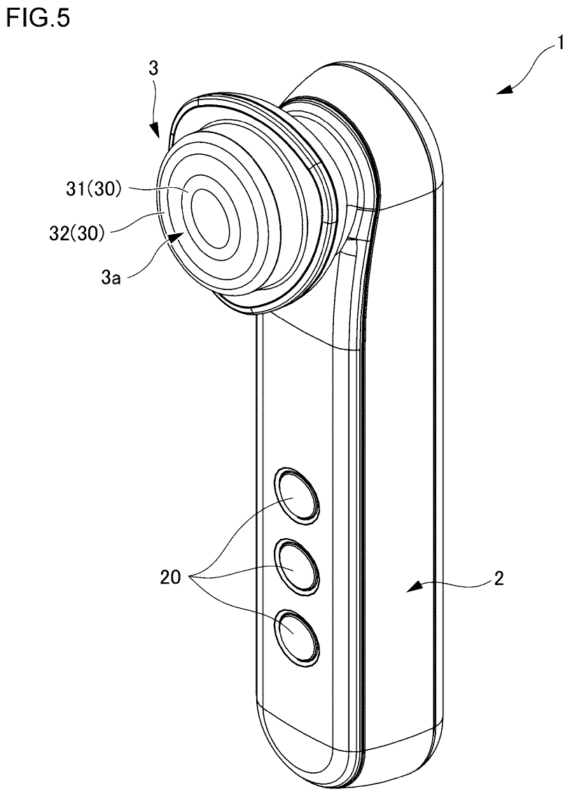

The electrode constitution shown in FIG. 1 to FIG. 4 is only an example. The electrode constitution is arbitrary as long as the output waveform of the AC stimulation of 40 kHz˜250 kHz described below can be applied to the user's skin. Therefore, for example, as shown in FIG. 5, a concentric two-annular electrode configuration, a concentric three-annular electrode configuration, an electrode configuration arranged in a straight line, a point-shaped electrode configuration with only one pair, and an electrode configuration including an annular electrode separated in the circumferential direction.

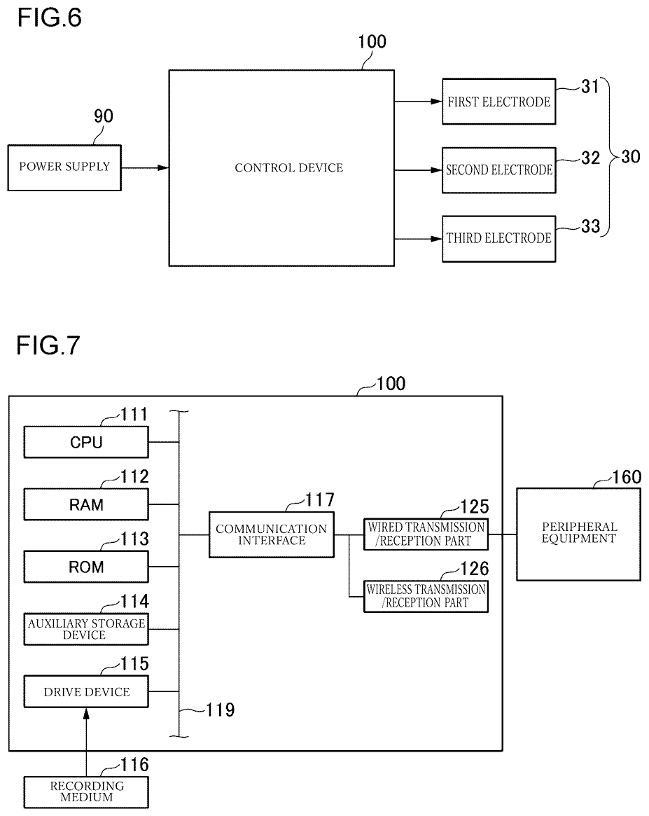

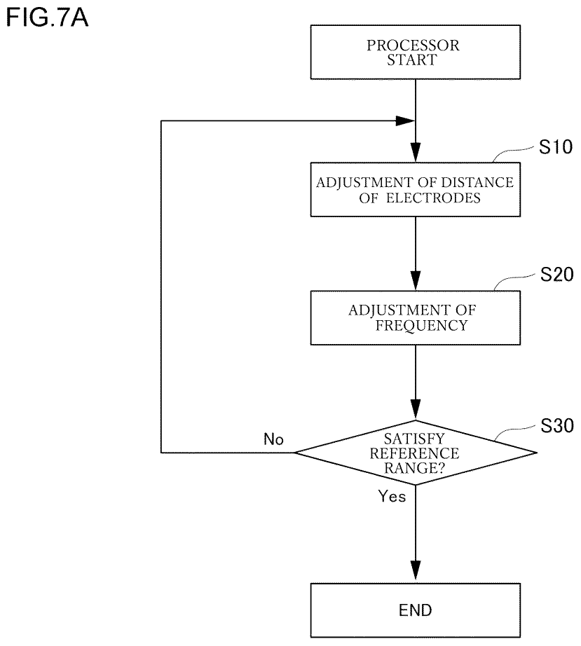

FIG. 6 is an explanatory diagram of a control device 100 built in the skin treatment device 1 according to this embodiment. FIG. 7 is schematically illustrating a peripheral equipment 160 related to a hardware configuration of the control devise 100.

The control device 100 is electrically connected to a power supply 90 and is electrically connected to the inner electrode 31, the outer electrode 32, and the outer edge electrode 33. The power supply 90 may be realized by an inner battery built in the skin treatment device 1 and/or an outer power supply connected to the skin treatment device 1. The control device 100 may a power supply circuit that generate various operating power supplies based on the power supply 90. And the control device 100 may include general-purpose processors, special-purpose processors, integrated circuits, ASICs (Application Specific Integrated Circuits).

In the example shown in FIG. 7, the control device 100 includes CPU (Central Processing Unit) 111, RAM (Random Access Memory) 112, ROM (Read Only Memory) 113, auxiliary storage device 114, drive device 115, and communication interface 117, which are connected by bus 119, and a wired transmission/reception part 125 and a wireless transmission/reception part 126 connected to the communication interface 117

The auxiliary storage device 114, which is for example HDD (Hard Disk Drive), SSD (Solid State Drive), is a storage device that stores data related to application software.

A wired transmission/reception part 125 includes a transmission/reception part that can communicate using a wired network. The peripheral equipment 160 is connected to the wired transmission/reception part 125. However, a part or/and all the peripheral equipment may be connected to a bus 119 or the wireless transmission/reception part 126. The peripheral equipment 160 may include the above-mentioned a plurality of electrodes 30 or for example portable devices such as user's smartphones. When portable devices are included, it may be possible that users set various setting related to the skin treatment device 1.

The wireless transmission/reception part 126 is a transmission/reception part that can communicate using a wireless network. The wireless network may include a mobile phone wireless communication network, the Internet, a VPN (Virtual Private Network), a WAN (Wide Area Network), and the like. Further, the wireless transmission/reception part 126 may include a near field communication (NFC: Near Field Communication) part, a Bluetooth (Bluetooth, registered trademark) communication part, a Wi-Fi (Wireless-Fidelity) transmission/reception part, an infrared ray transmission/reception part, and the like.

The control device 100 may be able to be connected to a recording medium 116. The recording medium 116 is an example of a readable storage medium, for example, is storing a predetermined program such as a program for realizing an output control method of the skin treatment device according to the present invention. The programs stored in this recording medium 116 is installed in the auxiliary storage device 114 of the control device 100 and the like via the drive device 115. The installed predetermined programs are realized by the CPU 111 of the control device 100. For example, the recording medium 116 may be a recording medium that records information optically, electrically or magnetically, such as a CD (Compact Disc)-ROM, a flexible disk, a magneto-optical disk, and the like, or a semiconductor memory that records information electrically, such as a ROM, flash memory, and the like. Note that the recording medium 116 does not include a carrier wave.

The control device 100 generates one or more types of output waveforms that can be applied to the skin via a plurality of electrodes 30 based on the power supply 90. And the program that can realize an output control method of the skin treatment device 1 according to the present invention is for example a program that operates a processor so as to adjust the frequency and the distance between the electrodes within a predetermined range. That is, the program according to the present invention may be a program that operates a processor so as to adjust the frequency and the distance between electrodes within a predetermined range, such that it simultaneously applies an electrical stimulation and a warming action. The distance between electrodes may be a distance between two or more electrodes, and a distance between a plurality of electrodes may be set in advance. For example, there may be only one pair of electrodes, and the program may adjust the frequency within a predetermined range according to the distance between the one pair of electrodes.

FIG. 7A is showing an example of the operation of the control device 100 according to this embodiment. FIG. 7 is showing an example of the operation of the control device 100 as processor. That is, FIG. 7 is an example showing an output control method of the skin treatment device according to the present invention.

In step S10 of FIG. 7A, the control device 100 adjusts a distance between a plurality of electrodes based on a predetermined program recorded on the recording medium 116 as mentioned above. The predetermined program is, for example, a program that realizes the output control method of the skin treatment device. The distance between a plurality of electrodes is, for example, the distance between a pair of electrodes. And the distance of the pair of electrodes is, for example, within a range of 0.5 mm˜60 mm. In case that the distance between electrodes is set in advance, step S10 is omitted, and the process proceeds to step S20.

In next step S20, the control device 100 adjust a frequency so that the skin treatment device applies a predetermined effect to the user's skin based on the predetermined program recorded in the record medium 116 as mentioned above. The predetermined effect is, for example, a predetermined AC stimulation that simultaneously applies an electrical stimulation and a warming action. The range of the frequency is, for example, 40 kHz˜250 KHz.

In step S30, when the control device 100 determines that the above mentioned adjustment result satisfies the reference range, that is, when “Yes” in FIG. 7A, it is determined that the adjustment is completed, and a control operation that simultaneously applies an electrical stimulation and a warming action is formed. On the other hand, in step S30, when the control device 100 determines that the above-mentioned adjustment result does not satisfies the reference range, that is, when “No” in FIG. 7A, the control device 100 returns to step S10, and performs the adjustment process again.

That is, the control device 100 according to the present invention controls the skin treatment device so as to simultaneously apply an electrical stimulation and a warming action to a user's skin surface by combining the AC stimulation of 40 kHz˜250 kHz with a distance between electrodes of 0.5 mm˜60 mm.

In this embodiment, the control device 100 generates an AC waveform MO (AC stimulation) having a frequency between 40 kHz˜250 kHz. In this case, the control device 100 generates the AC waveform MO so that it is possible to apply to the user's skin via the inner electrode 31 and the outer electrode 32. That is, the AC waveform MO can be applied to the user's skin with the inner electrode 31 as a positive electrode (or negative electrode) and the outer electrode 32 as a negative electrode (or positive electrode).

In this specification, unless otherwise stated, “AC waveform” is a concept that includes not only a sine wave, but also any waveform that has bipolarity.

In this embodiment, the AC waveform may be a rectangular wave, preferably, has a sinusoidal form, that is, has a form that gradually changes towards the peak value. In this case, the inconvenience that may occur when it is a square wave can be eliminated or reduced (for example, user discomfort due to a sudden increase in current).

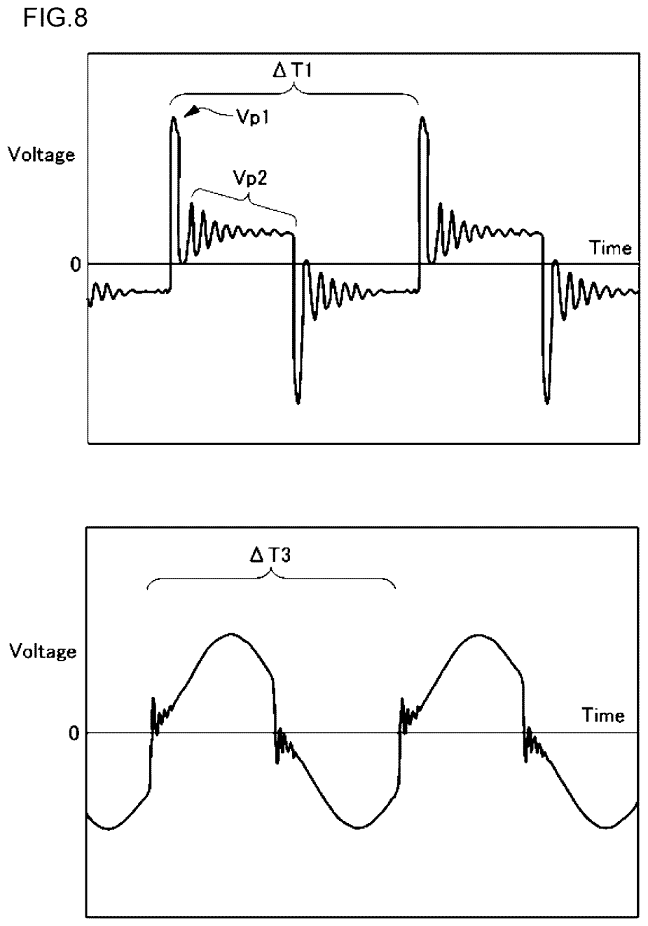

For example, the AC waveform may have a waveform shown in two examples in FIG. 8. FIG. 8 is showing the output waveform (time series waveform) of the AC waveform MO when time is taken on the horizontal axis and the voltage value is taken on the vertical axis. In FIG. 8, AT1 and AT3 represent an interval (range) corresponding to one cycle of an output waveform.

In this embodiment on the upper side of FIG. 8, the AC waveform has a plurality of peak voltage values during a half-cycle (ΔT/2). In this case, a plurality of peak voltage values include the first peak voltage value Vp1 and one or more second peak voltage value Vp2.

The first peak voltage value Vp1 is the peak voltage value that appears at the beginning of the half-cycle, the second peak voltage value Vp2 appears later than the first peak voltage value Vp1, and it is smaller than the first peak voltage value Vp1. A plurality of second peak voltages Vp2 may be generated in a manner that gradually decreases. The second peak voltage value Vp2 is preferably smaller than the half of the first peak voltage value Vp1.

The present inventor has found that by performing a test according to the following method using the frequency of the AC waveform MO and the distance between the electrodes as two parameters, when the values of these parameters are respectively within a predetermined range, a balancing of a warming effect and a muscle stimulation effect is effectively performed.

The test method is a sensory test, and Examinees are 11 people (N=11), and each of parameter is as follows.

A plurality of frequencies were set to 1 kHz, 10 kHz, 40 kHz, 70 kHz, 100 kHz, 165 kHz, 190 kHz, 250 kHz, and 300 kHz as follows.

A plurality of distances between electrodes were set to 0.5 mm, 1 mm, 1.8 mm, 2 mm, 3 mm, 5 mm, 10 mm, 20 mm, 30 mm, 40 mm, 50 mm, and 60 mm as follows.

Examinees gave the rating values related to the warmth feeling and the rating values related to a muscle stimulation according to rating reference as follows. As for the warmth feeling, “a rating value=1” means “feel nothing”, “the rating value=2” means “faintly warm”, “the rating value=3” means “sufficiently warm”, “the rating value=4” means “very warm”. And as for the muscle stimulation, “a rating value=1” means “feel nothing”, “the rating value=2” means “There is faint muscle stimulation”, “the rating value=3” means “There is sufficient muscle stimulation”, “the rating value=4” means “There is quite muscle stimulation”.

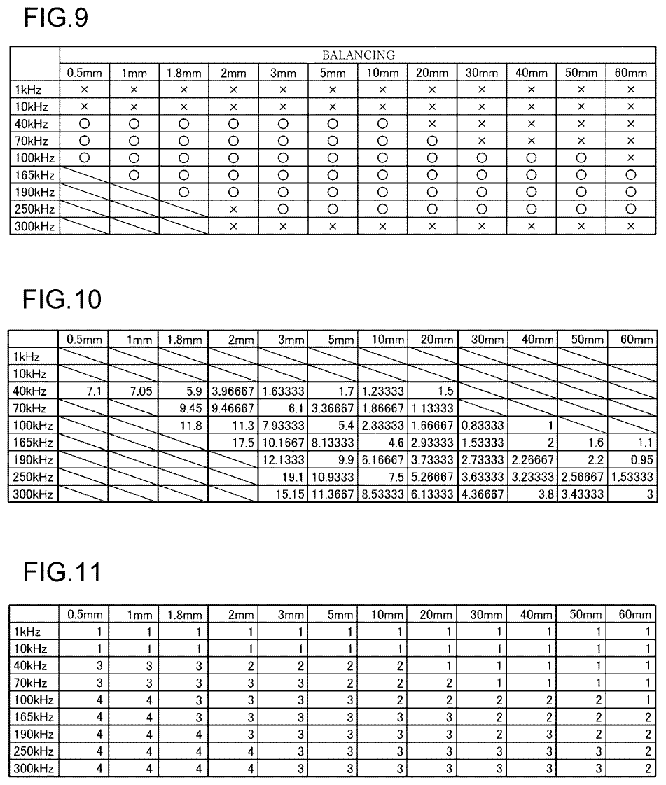

FIG. 9 is a table diagram showing a test result (rating result). In FIG. 9A “circle mark” indicates that the examinee's rating value for both warmth feeling and muscle stimulation is “2” or more. A “cross mark” indicates that the examinee's rating value for both warmth feeling and muscle stimulation is less than “2”. In addition, the “diagonal line” indicates that the temperature rise is relatively large and cannot be measured.

The result of sensory test related to the warmth feeling was consistent with a temperature measurement (actual measurement) result by thermography performed separately. FIG. 10 shows the temperature measurement (actual measurement) result by thermography, FIG. 11 shows the result of sensory test related to the warmth feeling. As can be seen in FIG. 10 and FIG. 11 when the rating value is “2” or more, temperature increase of 1 degree or more is indicated, and result that supports a reliability of the rating value for the warmth feeling is obtained. The measurement using thermography was performed on the skin region to which the AC stimulation of corresponding frequency was applied for 10 seconds.

In this way, by balancing an action of muscle stimulation and an action of warming, a best point of both actions is performed, and an action when a high frequency of 1 MHz or more or a low frequency of 1 kHz or less is applied can be realized by applying one frequency.

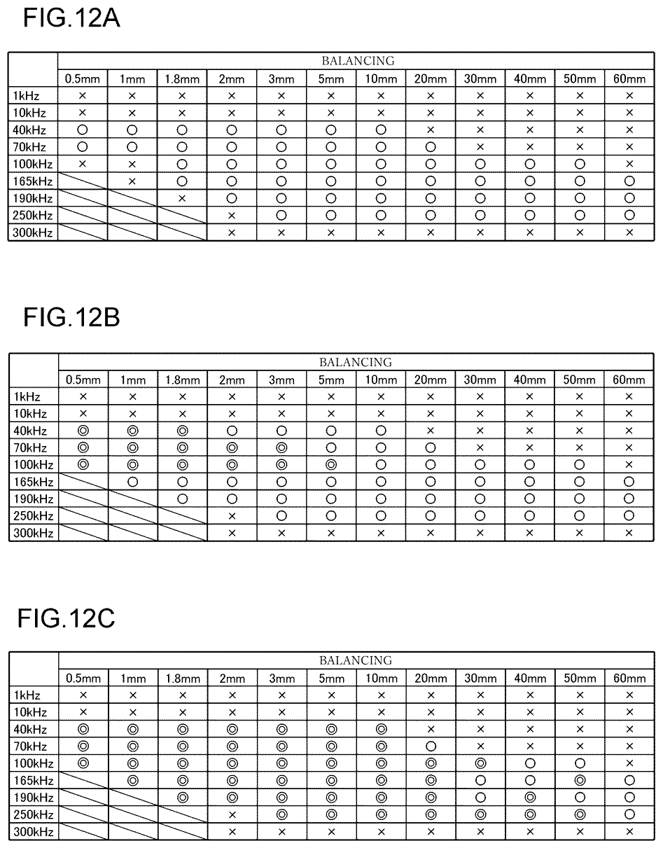

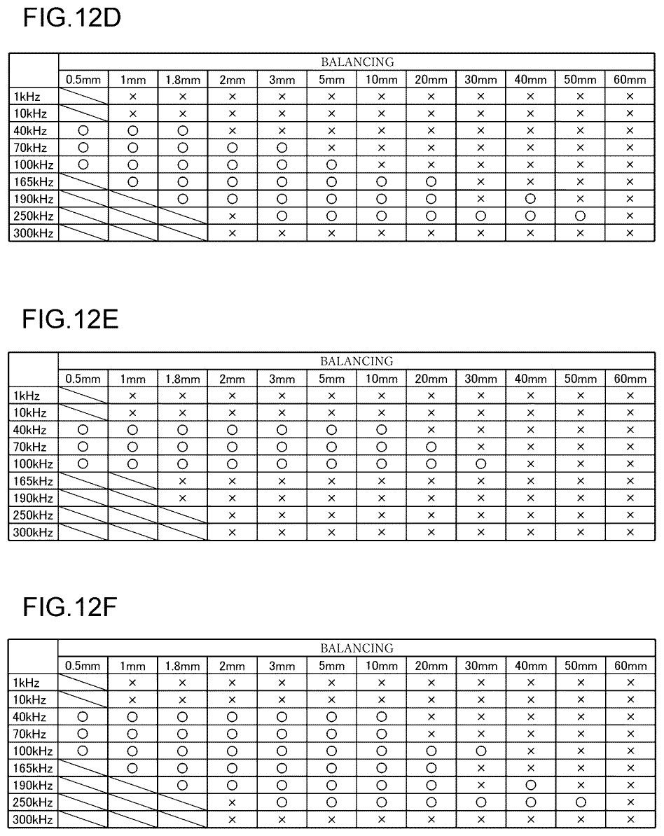

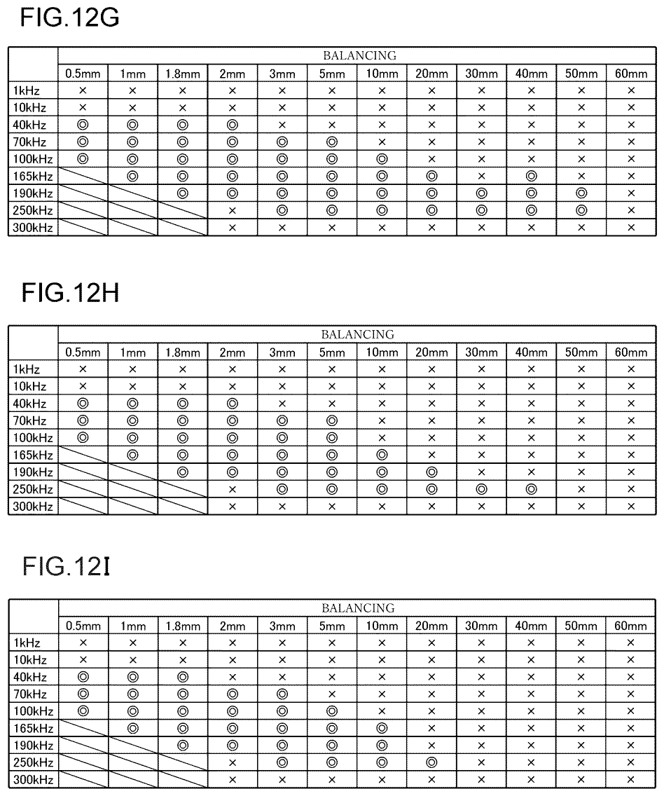

FIG. 12A to FIG. 12F are table diagrams indicating other classifications of test results (rating results).

In FIG. 12A a “circle mark” indicates when the rating value of the warmth feeling and the muscle stimulation respectively is “2” or “3”, a “cross mark” indicates other cases (that is, when the rating value of the warmth feeling and the muscle stimulation respectively is neither “2” nor “3”). A “diagonal line” indicates that a temperature rise is relatively high and unmeasurable.

In FIG. 12B, a “circle mark” indicates when the rating value of the warmth feeling and the muscle stimulation respectively is 2 or more, a “a double circle mark” indicates when the rating value of the warmth feeling and the muscle stimulation respectively is 3 or more, a “cross mark” indicates other cases. A “diagonal line” indicates that a temperature rise is relatively high and unmeasurable.

In FIG. 12C, a “circle mark” indicates when the rating value of the warmth feeling and the muscle stimulation respectively is 2 or more, a “a double circle mark” indicates when at least one of the rating values of the warmth feeling and the muscle stimulation is 3 or more, a “cross mark” indicates other cases. A “diagonal line” indicates that a temperature rise is relatively high and unmeasurable.

In FIG. 12D, “circle mark” indicates when the rating value of the warmth feeling is 3 or more and the muscle stimulation is 2 or more, a “cross mark” indicates other cases. A “diagonal line” indicates that a temperature rise is relatively high and unmeasurable.

In FIG. 12E, “circle mark” indicates when the rating value of the warmth feeling is 2 or more and the muscle stimulation is 3 or more, a “cross mark” indicates other cases. A “diagonal line” indicates that a temperature rise is relatively high and unmeasurable.

In FIG. 12F, a “circle mark” indicates when the rating value of the warmth feeling and the muscle stimulation respectively is 2 or more and at least one of the rating values of the warmth feeling and the muscle stimulation is 3 or more, a “cross mark” indicates other cases. A “diagonal line” indicates that a temperature rise is relatively high and unmeasurable.

FIG. 12G to FIG. 12J are table diagrams indicating classifications based on a combination of the rating results shown in FIG. 9 and the temperature measurement results by thermography.

In FIG. 12G, a “double circle mark” indicates when rating value of the examinees for both of warmth feeling and the muscle stimulation is 2 or more and a temperature rise measured by thermography is 2 degrees or more, a “cross mark” indicates when the rating value or the measurement results do not meet this case. A “diagonal line” indicates that a temperature rise is relatively high and unmeasurable.

In FIG. 12H, a “double circle mark” indicates when rating value of the examinees for both of warmth feeling and the muscle stimulation is 2 or more and a temperature rise measured by thermography is 3 degrees or more, a “cross mark” indicates when the rating value or the measurement results do not meet this case. A “diagonal line” indicates that a temperature rise is relatively high and unmeasurable.

In FIG. 12H, a “double circle mark” indicates when rating value of the examinees for both of warmth feeling and the muscle stimulation is 2 or more and a temperature rise measured by thermography is 4 degrees or more, a “cross mark” indicates when the rating value or the measurement results do not meet this case. A “diagonal line” indicates that a temperature rise is relatively high and unmeasurable.

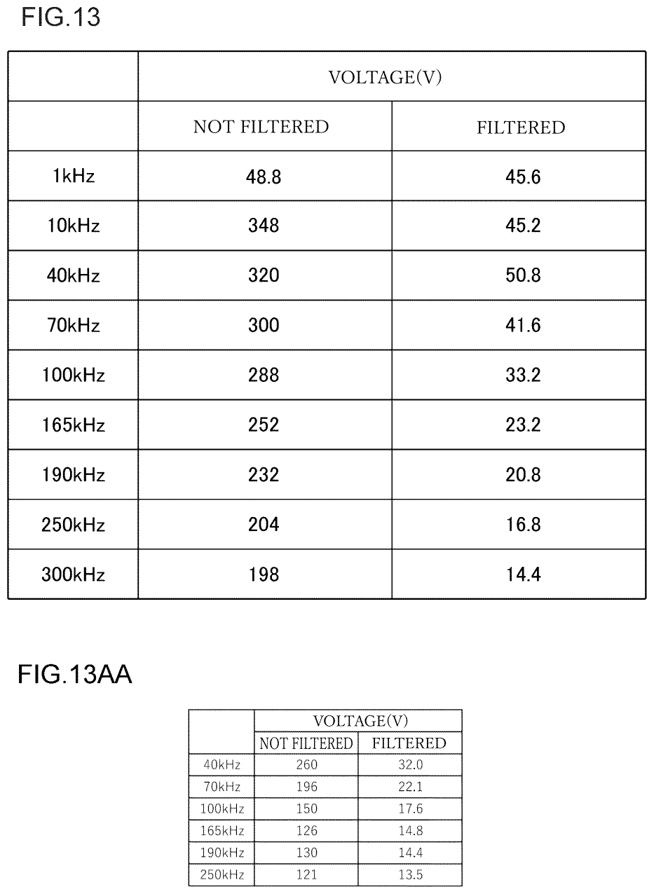

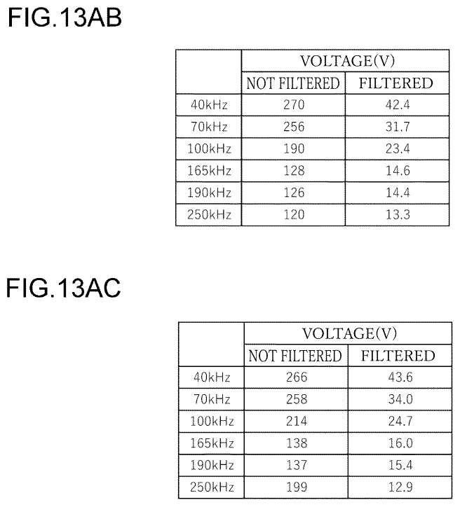

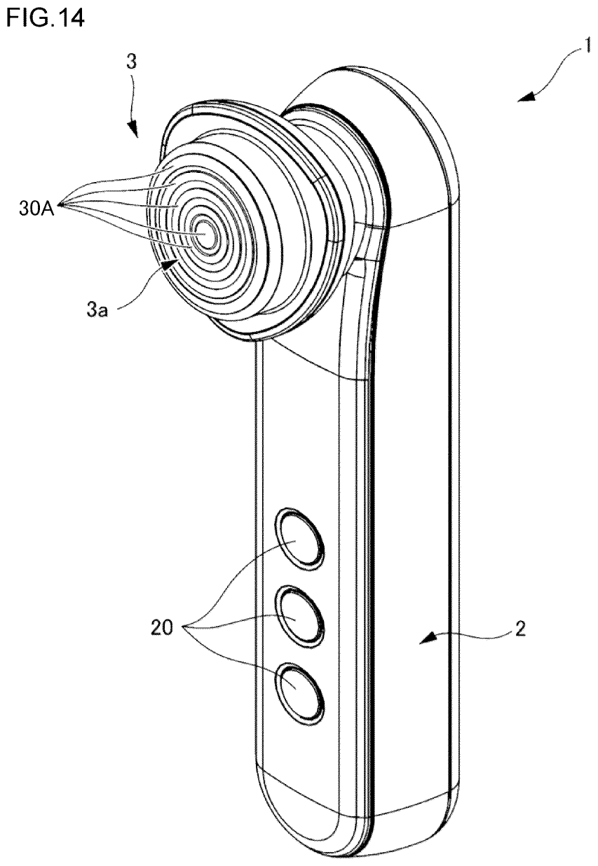

FIG. 13 indicates voltages used in this test. As shown in the “filtered” column in FIG. 13, relatively low voltages between 40V and 51V are used when less than 100 kHz, more lower voltages between 10V and 34V when 100 kHz or more.

Generally, the warmth feeling and the muscle stimulation are easily obtained when voltages are higher. In this embodiment, balancing a good warmth feeling and a muscle stimulation can be performed even when relatively low voltages in FIG. 13. It can be an effective and safe option without using high power consumption.

FIG. 13A indicates a voltage lower limit for balancing a warmth feeling and the muscle stimulation. FIG. 13AA indicates a voltage lower limit for balancing a warmth feeling and the muscle stimulation when the distance of electrodes is 0.5 mm. FIG. 13AB indicates a voltage lower limit for balancing a warmth feeling and the muscle stimulation when the distance of electrodes is 5 mm. FIG. 13AC indicates a voltage lower limit for balancing a warmth feeling and the muscle stimulation when the distance of electrodes is 10 mm. When the distance of electrodes is 0.5 mm, low voltages between 10V and 32V are used. When the distance of electrodes is 5 mm or 10 mm, low voltages between 10V and 44V are used. From the results of experimental data, when the distance of electrodes is 0.5 mm˜10 mm, it is suggested that balancing a warmth feeling and the muscle stimulation is well achieved even at a relatively low voltage. Generally, the warmth feeling and the muscle stimulation are easily obtained when the voltage is higher, however if the voltage is the lower limit voltage shown in FIG. 13AA to FIG. 13AC or more, it was confirmed that the balancing the good warmth feeling and the muscle stimulation was realized.

A muscle electrical stimulation induces muscle contraction by transdermal current and improves a thickness of muscle, and is effective for improvement of wrinkles, firmness, and sagging such like skin elasticity, sagging skin, lifting of double chin, and the like. In addition to this, an electrical warming stimulation warms the inside of the skin by transdermal current, so that it is effective for promoting blood flow and improving collagen production in skin fibroblast proliferation. Rather than overheating by warming the inside of the skin to an appropriate temperature (a temperature rise of 1 degree or more compared to before use and a warming state of 50° C. or less) and applying appropriate muscle electrical stimulation with a little pain, the user can use it continuously for a long time at a high frequency (2, 3, 4, 5, 6, 7 times a week).

An active ingredient for the skin is optional, for example, it may be as follows.

In pH levels in the vicinity of the weakly acidic range and neutral range

<Compounds that are Positively Charged in Water Solution, or Tend to be Positively Charged, or are Ampholytes>

Compounds that are positively charged or tend to be positively charged include tranexamic acid which is known to have a whitening effect, tranexamic acid derivatives such as cetyl tranexamate hydrochloride, and niacinamide, however are not limited to these. In addition, pyridoxine hydrochloride and its derivatives, which are said to be effective against acne and rough skin, benzalkonium chloride, which is used for sterilization and disinfection, and peptides with an isoelectric point on the alkaline side, such as palmitoyl tripeptide-5, acetyl hexapeptide-8, and dipeptide diaminobutyroyl benzylamide diacetate, are also included. Further examples include allantoin, aldioxa, carnitine HCl, basic amino acids such as lysine, arginine, histidine, tryptophan, ornithine, and further ergothioneine, and urea as a moisturizing agent. However, any substance having a functional group that is positively charged or polarized at a pH level in the vicinity of weakly acidic to weakly alkaline (the amount of charge may be minute, as long as it is cationic) may be used, and the present invention is not limited to these compound groups.

In addition, the ampholytes having a functional group that is positively charged or polarized at a pH level in acidic to weakly alkaline include tranexamic acid, glycine, proline, alanine, serine, Acetyl Hydroxyproline, ¿-aminocaproic acid, neutral amino acids such as γ-aminobutyric acid and derivatives thereof, trimethyl glycine, and the like.

<Compounds that are Negatively Charged in Water Solution, or Tend to be Negatively Charged>

In addition, potassium salt 4-methoxysalicylate and disodium adenosine monophosphate, which are recognized as effective whitening agents, ascorbic acid, L-ascorbic acid 2-glucoside, L-ascorbic acid sodium phosphate, L-ascorbic magnesium phosphate, L-ascorbic acid sulfate ester disodium acid, ascorbic acid and derivatives thereof such as trisodium ascorbyl Palmitate Phosphate, sodium dl-α tocopheryl phosphate, and the like. In addition, zinc paraphenol sulfonate, salicylic acid and its sodium salt, sodium lactate, sodium L- or DL-pyrrolidone carboxylic acid solution, acidic amino sodium such as sodium L-glutamate, sodium L-aspartate are included. In addition, glycyrrhizinic acid which is said to have the effect of calming inflammation, glycyrrhizic acid and salts thereof such as dipotassium glycyrrhizinate and ammonium glycyrrhizinate, sodium guaiazurene sulfonate, sodium lysine dilauroyl glutamate, and the like. It may be a substance having a functional group that is negatively charged or polarized at a pH near weakly acidic˜weakly alkaline (the amount of charge may be anionic even if it is small), and it is not limited to the above.

<Compounds that are Hardly Charged in Water Solution>

Kojic acid, arbutin, hydroquinone, 4-(1-phenylethyl)-1,3-benzenediol, 4-n-butylresorcinol, 5,5′-dipropyl-biphenyl-2,2′-diol, ellagic acid, 3-O-ethyl ascorbic acid, 3-glyceryl ascorbic acid, bisglyceryl ascorbic acid, hexyl 3-glyceryl ascorbic acid, myristyl 3-glyceryl ascorbic acid, ascorbic acid derivatives such as 3-lauryl glyceryl ascorbic acid, D-pantothenyl alcohol, cholecalciferol, 3-O-cymen-5-ol (isopropylmethylphenol), sugars such as xylose, sorbitol, mannitol, and butylene glycol, hexylene glycol, pentylene glycol, polyols such as glycerin, and terpenes such as hinokitiol. In addition, fullerene which is poorly soluble substance, oryzanol, ceramide EOP, ceramide EOS, ceramide NG, caprooyl sphingosine, ceramide NP, N-stearoylphytosphingosine, N-stearoyl phytosphingosine, N-stearoyl dihydrosphingosine, ceramide AG, ceramide AP, hydroxystearyl phytosphingosine, ceramide 6II, phytosphingosine are also included as useful components regardless of whether these are contained in liposomes or not. Examples thereof include extracts obtained from plants and animals that perform usefulness, culture mediums such as stem cells, and culture supernatant.

In addition, as the beaty ingredients that dissolve in coexistence with water-soluble or water-soluble solvents, and as beauty ingredients that dissolve as micelles in the aqueous phase, flavonoid includes isoflavones, licorice root extract, licorice flavonoids, licorice flavonoids, and the like, but it is not limited to the case. As Extracts chamomilla ET, clara root extract, sembri extract, carrot and its root extract, soybean extract and soybean seed extract, tea leaf extract, galactomyces culture medium, rice power No. 11 (rice extract No. 11), astaxanthin solution and red algae extract, placenta extract, placenta extract (1)˜(5), and water-soluble and hydrolyzed placenta extracts and the like are included.

### <Lipids and Oil-Soluble Substances>

Squalane, linoleic acid, ascorbyl tetra 2-hexyldecanoate, ascorbyl dipalmitate, retinol, retinol acetate, retinol palmitate, hydrogenated retinol, retinol and derivatives thereof such as retinol linoleate and the like, tocopherol nicotinate, dl-α-tocopherol, d-8-tocopherol, natural vitamin E, tocopherol and the derivatives thereof such as DL-α-tocopherol acetate, stearyl glycyrrhetinate, estradiol, ethinyl estradiol, astaxanthin, rice germ oil, phospholipid such as squalane including being botanical or synthetic, guaiazulene and guaiazulene sulfonate esters, ascorbyl stearate, fatty acid esters of ascorbic acid such as ascorbyl palmitate, lauroyl glutamic acid di (phytosteryl/octyldodecyl), oil-soluble placenta, and the like are included.

<Compounds with Relatively High Molecular Weight and Macromolecule Compounds>

Human genetically modified oligopeptide-1, palmitoyl hexapeptide including palmitoyl hexapeptide-4, palmitoyl pentapeptide, hydrolyzed collagen and derivatives thereof, hyaluronic acid, sodium hyaluronate, hyaluronic acid and the derivatives thereof such as acetylated sodium hyaluronate, white jellyfish polysaccharide, alcaligenes polysaccharide, and polyquaterniums are included.

### (Another Embodiment)

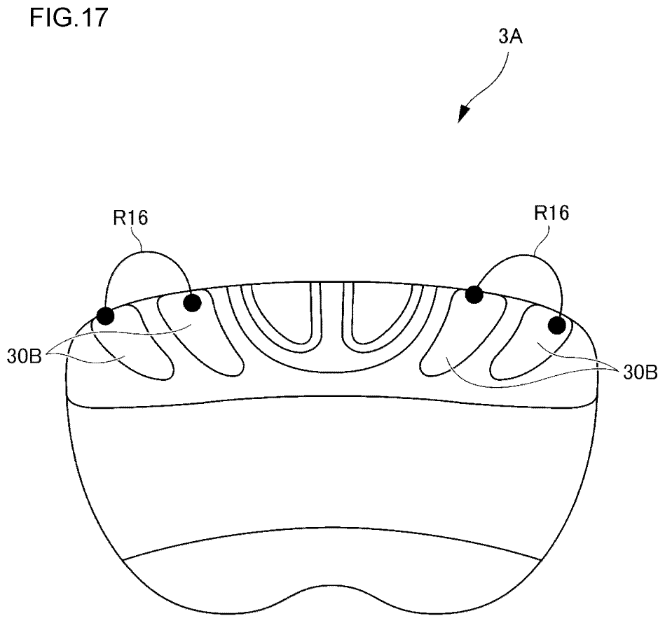

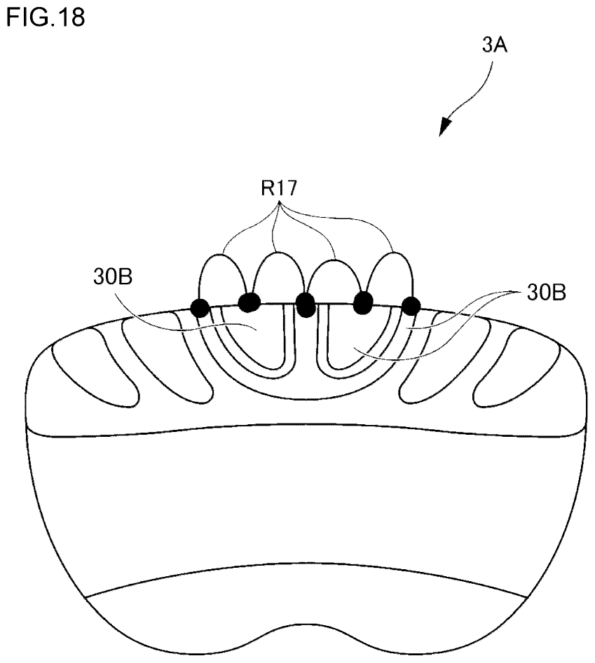

FIG. 14 to FIG. 18 are explanatory diagrams of the electrodes according to another embodiment. FIG. 14 indicates a plurality of electrodes 30A arranged in an annular ring-shape around the center C of the contact surface 3a of the head part 3A. In FIG. 14, five electrodes 30A are shown, however a quantity of the electrode 30A is optional as long as it is possible to apply the output waveform of above-mentioned AC stimulation of 40 kHz˜250 kHz to user's skin. The distance between respective electrodes 30A may be same in radial direction or different significantly. The distance between respective electrodes 30A is correspond to the difference between an outer diameter of inner electrode based on the center C and the inner diameter of the outer electrode adjacent to the electrode when a plurality of electrodes 30A are sequentially arranged from the center C to outer.

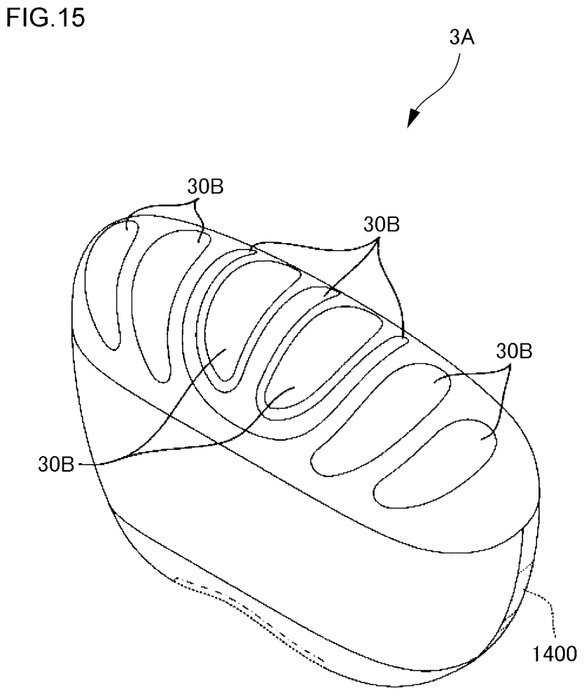

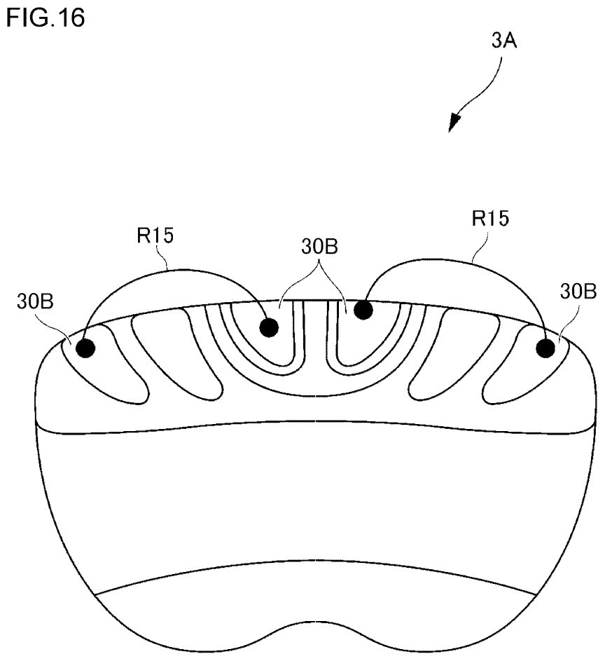

FIG. 15 shows an arrangement of the electrodes 30A in the head part 3A, FIG. 16 to FIG. 18 shows variations (modes) of relationships of a pair of electrodes when the electric stimulation is applied. The distance between electrodes may be set to increase variations. In the example in FIG. 15, a removable cover 1400 that covers a terminal for USB connection is illustrated.

With such an electrode structure, various distances of electrodes can be provided by combinations of the pair of electrodes. FIG. 16 to FIG. 18 show variations of the pair of electrodes. As shown in FIG. 14 to FIG. 18, The present invention can suitably perform balancing between the electric stimulation and the muscle stimulation by properly controlling the frequency even if it implements a structure that can applies the electric stimulation by the various distances of electrodes.

It should also be noted that the present applicant is only aware of the literature known invention described in the literature of “prior art literature” column herein, and the present invention is not necessarily intended to solve the problems in the literature known invention. The problems to be solved by the present invention should be acknowledged by considering all the specification. For example, in the present specification, when there is a description that a predetermined effect is achieved by a specific configuration, a problem that is the flip side of the predetermined effect can be solved. However, it is not necessarily the intention to make such a specific configuration a mandatory requirement.

Although each embodiment has been described in detail above, it is not limited to a specific embodiment, and various modifications and changes are possible within the scope described in the claims. It is also possible to combine all or a plurality of the components of the above-described embodiments.

### LIST OF REFERENCE SIGNS

1 skin treatment device
2 holding part
3 head part
3a contact surface
20 user interface
30 electrode
31 inner electrode
32 outer electrode
33 outer edge electrode
90 power supply
100 control device
111 CPU
112 RAM
113 ROM
114 auxiliary storage device
115 drive device
116 recording medium
117 communication interface
119 bus
125 wired transmission/reception part
126 wireless transmission/reception part
160 peripheral equipment

1. A method for skin treatment, comprising:
simultaneously applying an electric stimulation and a warming action to a skin surface by combining an AC (alternating current) stimulation of 40 kHz˜250 kHz with a distance between electrodes of 0.5 mm˜60 mm related to a pair of the electrodes that apply the AC stimulation to skin.

2. The method for skin treatment according to claim 1, wherein
simultaneously applying the electric stimulation and the warming action to the skin surface by combining the AC stimulation of 40 kHz˜190 kHz with the distance between electrodes of 1.8 mm˜10 mm related to the pair of electrodes that apply the AC stimulation to skin.

3. The method for skin treatment according to claim 1, wherein
simultaneously applying the electric stimulation and the warming action to the skin surface by combining the AC stimulation of 40 kHz˜100 kHz with the distance between electrodes of 0.5 mm˜10 mm related to the pair of electrodes that apply the AC stimulation to skin.

4. A skin treatment device, comprising:
a control device that simultaneously applies an electric stimulation and a warming action to a skin surface by combining an AC (alternating current) stimulation of 40 kHz˜250 kHz with a distance between electrodes of 0.5 mm˜60 mm related to a pair of electrodes that apply the AC stimulation to skin.

5. An output control method of a skin treatment device, comprising:
controlling so as to simultaneously apply an electric stimulation and a warming action to a skin surface by combining an AC (alternating current) stimulation of 40 kHz˜250 kHz with a distance between electrodes of 0.5 mm˜60 mm related to a pair of electrodes that apply the AC stimulation to skin.

6. A readable storage medium storing a program causing a skin treatment device to realize an output control method of the skin treatment device, wherein the output control method, comprising:
controlling so as to simultaneously apply an electric stimulation and a warming action to a skin surface by combining an AC (alternating current) stimulation of 40 kHz˜250 kHz with a distance between electrodes of 0.5 mm˜60 mm related to a pair of electrodes that apply the AC stimulation to skin.
